(19) United States
(12) Patent Application Publication
(10) Pub. No.: US 2026/0338711 A1
(43) Pub. Date: Oct. 29, 2026

(54) SYSTEM FOR AUTOMATIC MAPPING OF GOLF COURSE SOIL MOISTURE

(71) Applicant: TERRARAD TECH AG, Zürich (CH)

(72) Inventor: Derek HOUTZ, Zürich (CH)

(21) Appl. No.: 19/118,947

(22) PCT Filed: May 12, 2023
(86) PCT No.: PCT/CH2023/050016

§ 371 Date: Apr. 7, 2025

(30) Foreign Application Priority Data
Oct. 10, 2022 (CH) CH001189/2022

(51) Int. Cl. G01N 22/04; A01G 25/16; G01C 21/00; G01N 33/24

(52) U.S. Cl. CPC G01N 22/04; A01G 25/167; G01C 21/3848; G01N 33/246

(57) ABSTRACT

The disclosed invention provides means of measuring soil moisture on a golf course over a wide area and to provide guidance for irrigation. This is reached by a system for automatic mapping of golf course soil moisture, characterized in that, such system comprises at least one portable microwave sensor mounted by a sensor mounting device to a golf course turfgrass vehicle operated by a golf course operator, while a global navigation satellite system, a wireless transmission system and a central server are operatively connected to the at least one portable microwave sensor such, that after data collection and analysis spatially resolved maps of soil moisture can be output to the operator of the golf course.

### TECHNICAL FIELD

The present invention describes a system for automatic mapping of golf course soil moisture, use of at least one portable microwave sensor mounted with a sensor mounting device to a golf course turfgrass vehicle and method for automatic mapping of golf course soil moisture and preparation of area-wide irrigation instructions of a golf course.

### STATE OF THE ART

Golf courses have different areas, such as tees, fairways and greens with holes, each of which is covered with vegetation consisting of different types of grass. In addition to shrubs and woods, bunkers are also embedded in the landscape in particular filled with sand. Above all, the lawns necessary for the game of golf must have sufficient, but not too high, soil moisture in order to create optimal playing conditions for the golf player.

Today, the soil moisture in the various areas of the golf course is monitored by so-called greenkeepers and adjusted to target values. The greenkeeper has special knowledge for the highly complex maintenance of lawns, especially for preparation for professional tournaments.

The different lawns require different but usually considerable amounts of irrigation. The greenkeeper should therefore measure the soil moisture in each case before implementing a watering schedule. Soil conditions that are too wet or too dry are not optimal for golf, and both extremes can damage the turf in the long run.

In addition to the goal of optimal soil moisture and turf quality, attention must also be paid to the consumption of resources. Efficient use of irrigation water is also a concern, and knowledge of soil moisture helps to reduce water consumption and the cost of irrigation on golf courses. All this is today done manually by the greenkeeper, using electronic measuring devices to measure the soil moisture, which devices are inserted manually in places into the ground. From this, the greenkeeper can roughly determine the soil moisture in different sections of the golf course.

Moisture monitoring and setting up of irrigation schedules are done entirely by greenkeepers, while automatic sprinkler systems do the watering as set by the greenkeeper. So, a lot of knowledge about the golf course is needed, about the spot measurement of soil moisture, and the operation of the irrigation systems.

The previous procedure is cumbersome and has not led to area-wide soil moisture determinations, so that the lawn cannot be optimally maintained.

### DESCRIPTION OF THE INVENTION

The object of the present invention is to provide a means of measuring soil moisture on a golf course over a wide area at a high resolution to provide guidance for irrigation.

This is done by the use of a microwave sensor mounted to a golf course mower, and a method for the preparation of area-wide irrigation instructions of a golf course.

### BRIEF DESCRIPTION OF THE DRAWINGS

Further understanding of various aspects of the invention can be obtained by reference to the following detailed description in conjunction with the associated drawings, which are described briefly below.

It should be noted that in the differently described embodiments, the same parts are provided with the same reference symbols or the same component names, the disclosures contained in the entire description being able to be applied analogously to the same parts with the same reference symbols or the same component symbols.

The subject matter of the invention is described below in conjunction with the attached drawings.

FIG. 1 shows a perspective view of a typical scene on a golf course, while

FIG. 2 shows a schematic view of the disclosed system, comprising a golf course turfgrass vehicle with mounted portable microwave sensor, wireless connected to a central server.

### DESCRIPTION

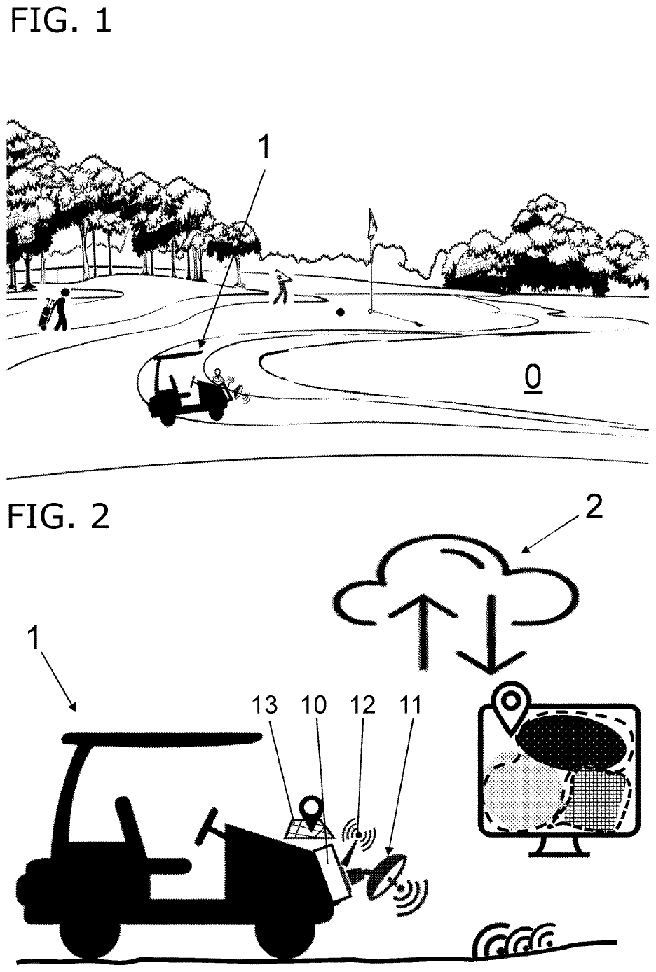

FIG. 1 shows a typical scene on a golf course 0, with golf player and a golf course turfgrass vehicle 1. Such golf course turfgrass vehicle 1 can be a grass cutting vehicle or mower, manned or unmanned robotic, a maintenance vehicle, or a golf players' golf cart 1, which is usually already in use on a golf course 0 respectively operated by a golf course operator.

We have sought a way to implement an automatic method of measuring soil moisture and mapping soil moisture on the golf course 0, while the golf course 0 is used, and without requiring additional labor. For this purpose, the golf course turfgrass vehicle 1 is equipped with various additional means.

In an addition, after creating maps of soil moisture in different areas, a determination of applicable and optimal watering schedules of the golf course 0 can also be calculated.

It is desirable for water savings and golf course playability to have uniform soil water content, and accurate, high resolution data in time and space on the soil moisture. Many golf courses 0 have the ability to precisely control irrigation or sprinkler systems allowing fine spatial control of water application, but they normally do not have the mapping of golf course soil moisture.

As shown in FIG. 2 the golf course turfgrass vehicle 1 comprises a sensor mounting device 10, a portable microwave sensor 11, a wireless transmission system 12 and a global navigation satellite system 13. All parts are usually controlled by a non-depicted electronics.

The mounting device 10 and the portable microwave sensor 11 are most preferred mounted forward-looking or rear-looking, but also side-looking mountings are possible. The mounting device 10 is a mechanical mounting system such that the portable microwave sensor 11 is sufficiently fixed with a clear view onto the golf course 0 surface, while the golf course turfgrass vehicle 1 drives across the golf playing surfaces. Most preferred the mounting device 10 should be an easily detachable device allowing it to attach to various golf course turfgrass vehicles.

Mower-mounted passive microwave sensors are optimal for measurement of soil water content under turfgrass on golf courses.

As portable microwave sensors 11, passive microwave sensors are known as microwave radiometers, which can be used for soil water content, soil moisture or soil humidity sensing. Such passive microwave sensors are using microwave frequencies in a frequency range 300 MHz-8 GHz in P-band and/or more preferred in L-band (SMOS and SMAP) in the passive-protected frequency range from 1400-1427 MHz and/or in the C-band can be used (AMSR, AMSR-E, AMSR2) with frequencies in the range from 6-8 GHz.

As portable microwave sensors 11, also active microwave sensors such as radar or Synthetic Aperture Radar (SAR) systems, most preferred operating in the L-, C- and/or X-bands, can be used. Such portable radar systems or ground-penetrating radars, can sense soil moisture measuring reflected backscatter in the frequency range 300 MHz-8 GHz.

Microwave sensing of the surface of a golf course 0, whether it be active or passive, is optimal for this purpose due to its deep effective measurement depth (~1-50 cm) in soil. Passive sensors show relative insensitivity to vegetation structure when compared to active microwave soil sensing techniques. Whereas active sensors have relatively less sensitivity to external transmissions and radio-frequency interference (RFI) sources. Both active and passive microwave sensors 11 have their advantages for remote sensing of soil moisture.

The wireless transmission system 12 can automatically upload measured data from the portable microwave sensor 11 to a central server 2, most preferred with processing means, while such central server 2 can be local or a cloud. Data dispatch is done with known data dispatch protocols. The server 2 can be also fixed to the vehicle 1 or be separated at a place on the golf course 0 areal or can be reachable via internet in the cloud.

The global navigation satellite system 13, known in the art, like most generic GNSS receiver or NAVSTAR GPS or Galileo or others, geolocates the readings taken by the portable microwave sensor 11 and includes this geolocation information in the measured microwave data uploaded by the wireless transmission system 12.

In practice the portable microwave sensor 11 is mounted to the golf course turfgrass vehicle 1 and turns on with the golf course turfgrass vehicle 1, and begins collecting single, dual, or full polarization brightness temperature and/or backscatter measurements of the turfgrass scene, as well as of internal calibration sources.

Usually, the golf course turfgrass vehicle 1 makes systematic passes over the fairways, and other designated playing areas of the golf course 0, like tee boxes and rough with passes being spaced by the width of the golf course turfgrass vehicle 1, about one to three meters typically for a fairway mower 1. Typically, the portable microwave sensor 11 records samples together with samples of the sensor's geolocation with the global navigation satellite system 13 multiple times per second. This corresponds to multiple measurements per meter in the direction of motion for typical golf course turfgrass vehicle 1 driving speeds of a few meters per second.

The generated spatial map of soil moisture thus has spatial resolution approximately equal to the width of the golf course turfgrass vehicle 1 in the direction orthogonal to golf course turfgrass vehicle 1 motion and sub-meter resolution in the direction of motion. Geolocation accuracy of the resulting map is also governed by the accuracy of the global navigation satellite system 13 in use (GNSS, GPS, etc.).

Data is most preferred transmitted wirelessly in real-time but could also be transferred upon completion of driving procedure to the central server 2 with processing means, where data can be processed, interpolated, displayed, and fed into irrigation programming functions.

If mower 1 of a golf course 0 are used as golf course turfgrass vehicle 1, the data from multiple mower-mounted portable microwave sensors 11 can be combined in the central server 2, because typical golf courses 0 use a number of mowers 1 in parallel to cover more area per time. Mowers 1 are typically already traversing the playing surfaces of the golf course 0 on an at-least weekly basis, providing an opportune platform for high resolution data generation.

A processing software installed and running on the central server 2, which is local or in the cloud, receives the transmissions by wireless transmission system 12, comprising measurements of portable microwave sensor 11 and geolocation information from global navigation satellite system and compiles readings into a map of soil water content, soil moisture or soil humidity of the golf course 0.

The processing software may be used to interpolate these many point measurements to a standardized grid for display, assemble daily, weekly and average soil water content maps of the golf course 0.

The processing software may compile statistics of each course grass type (fairway, rough, tee boxes). The processing software may also directly suggest irrigation (watering) amount on a per sprinkler basis and generate watering prescriptions to feed the watering plan directly. The processing software uses the maps to automatically program not depicted irrigation systems according to the generated data. For example, sprinklers of the irrigation system in regions with high soil water content will be prescribed less watering time and/or water volume, whereas sprinklers in regions with relatively low soil water content will be programmed to apply more water.

Access to the server 2 with measurement data and calculated data, like mappings and irrigation schedules is provided by computational means like personal computer or smart devices.

### LIST OF REFERENCE NUMERALS

0 golf course
1 golf course turfgrass vehicle
10 sensor mounting device
11 portable microwave sensor (passive (radiometer) or active (radar))
12 wireless transmission system
13 global navigation satellite system (GNSS receiver/NAVSTAR GPS or Galileo or . . . )
2 central server (local or cloud) with processing means
Processing software (APP) for data evaluation, map production, irrigation instructions
Please amend the claims as follows:

1. A system for automatic mapping of golf course soil moisture, wherein such system comprises at least one portable microwave sensor mounted by a sensor mounting device to a golf course turfgrass vehicle operated by a golf course operator, while a global navigation satellite system, a wireless transmission system and a central server are operatively connected to the at least one portable microwave sensor such, that after data collection and analysis spatially resolved maps of soil moisture can be output to the operator of the golf course.

2. The system according to claim 1, wherein the sensor mounting device is a mechanical mounting system for sufficient fixation of the portable microwave sensor, permitting a clear view onto the golf course surface and most preferred designed to be easily removable.

3. The system according to claim 1, wherein the at least one portable microwave sensor is a passive microwave sensor or microwave radiometer, using microwave frequencies in a frequency range 300 MHz-8 GHz, most preferred working in L-band (SMOS and SMAP) in the passive-protected frequency range from 1400-1427 MHz and/or in the C-band can be used (AMSR, AMSR-E, AMSR2) with frequencies in the range from 6-8 GHz.

4. The system according to claim 1, wherein the wireless transmission system is operatively connected to the central server with processing means to automatically upload measured data from the portable microwave sensor to the central server with known data dispatch protocols.

5. The system according to claim 1, wherein a processing software for data recording, sending, analysis, map creation and/or map display is installed and running on the central server, which can be local or a cloud.

6. A method comprising:
using at least one portable microwave sensor mounted with a sensor mounting device to a golf course turfgrass vehicle a wireless transmission system, a global navigation satellite system and a central server, which are operatively connected to the at least one portable microwave sensor for spatially resolved automatic recording of soil moisture data in areas of a golf course combined with geolocation information from global navigation satellite system and determination of maps of soil moisture on the golf course, controlled by a processing software.

7. The method according to claim 6, wherein additionally to the determination and mapping of soil moisture a guidance for later irrigation in form of a watering schedule of the golf course areas is provided after analysis in the central server via the processing software.

8. The method according to claim 6, wherein at least one portable microwave sensor is mounted with the sensor mounting device to a grass cutting vehicle or mower or a golf players' golf cart, automatically recording and sending data, while the vehicle starts moving.

9. The method according to claim 6, wherein the at least one portable microwave sensor is a passive microwave sensor or microwave radiometer, working in L-band (SMOS and SMAP) in the passive-protected frequency range from 1400-1427 MHz and/or in the C-band can be used (AMSR, AMSR-E, AMSR2) with frequencies in the range from 6-8 GHz.

10. A method for automatic mapping of golf course soil moisture and preparation of area-wide irrigation instructions of a golf course,
wherein
at least one portable microwave sensor mounted by a sensor mounting device to a golf course turfgrass vehicle is moved across a golf course, while
measurements with the at least one portable microwave sensor are automatically done,
connected with geolocation information from a global navigation satellite system; and
all data are sent via a wireless transmission system to a central server, controlled by processing software installed and running on the central server, resulting in
determination of maps of soil moisture on the golf course, controlled by the processing software, which can be accessed on the central server.

11. The method according to claim 11, wherein additionally to the determination and mapping of soil moisture of the golf course, a guidance for later irrigation in form of a watering schedule of the golf course areas is provided after analysis in the central server via the processing software.

12. The system according to claim 2, wherein the at least one portable microwave sensor is a passive microwave sensor or microwave radiometer, using microwave frequencies in a frequency range 300 MHz-8 GHz, most preferred working in L-band (SMOS and SMAP) in the passive-protected frequency range from 1400-1427 MHz and/or in the C-band can be used (AMSR, AMSR-E, AMSR2) with frequencies in the range from 6-8 GHz.

13. The system according to claim 2, wherein the wireless transmission system is operatively connected to the central server with processing means to automatically upload measured data from the portable microwave sensor to the central server with known data dispatch protocols.

14. The system according to claim 3, wherein the wireless transmission system is operatively connected to the central server with processing means to automatically upload measured data from the portable microwave sensor to the central server with known data dispatch protocols.

15. The system according to claim 2, wherein a processing software for data recording, sending, analysis, map creation and/or map display is installed and running on the central server, which can be local or a cloud.

16. The system according to claim 3, wherein a processing software for data recording, sending, analysis, map creation and/or map display is installed and running on the central server, which can be local or a cloud.

17. The system according to claim 4, wherein a processing software for data recording, sending, analysis, map creation and/or map display is installed and running on the central server, which can be local or a cloud.

18. The method according to claim 7, wherein at least one portable microwave sensor is mounted with the sensor mounting device to a grass cutting vehicle or mower or a golf players' golf cart, automatically recording and sending data, while the vehicle starts moving.

19. The method according to claim 7, wherein the at least one portable microwave sensor is a passive microwave sensor or microwave radiometer, working in L-band (SMOS and SMAP) in the passive-protected frequency range from 1400-1427 MHz and/or in the C-band can be used (AMSR, AMSR-E, AMSR2) with frequencies in the range from 6-8 GHz.

20. The method according to claim 8, wherein the at least one portable microwave sensor is a passive microwave sensor or microwave radiometer, working in L-band (SMOS and SMAP) in the passive-protected frequency range from 1400-1427 MHz and/or in the C-band can be used (AMSR, AMSR-E, AMSR2) with frequencies in the range from 6-8 GHz.
